(19) United States
(12) Patent Application Publication
(10) Pub. No.: US 2025/0115920 A1
(43) Pub. Date: Apr. 10, 2025

(54) HOST CELLS AND METHODS USEFUL FOR PRODUCING CALCIUM PHOSPHATE BASED COMPOSITE BIOMATERIALS

(71) Applicant: THE REGENTS OF THE UNIVERSITY OF CALIFORNIA, Oakland, CA (US)

(72) Inventors: Isaak Elis MUELLER, Cambridge, MA (US); Yasuo YOSHIKUNI, Orinda, CA (US); Yu-Wei LIN, Berkeley, CA (US); Peter ERCIUS, Orinda, CA (US)

(21) Appl. No.: 18/915,066

(22) Filed: Oct. 14, 2024

Related U.S. Application Data
(63) Continuation of application No. PCT/US2023/018715, filed on Apr. 14, 2023.
(60) Provisional application No. 63/331,738, filed on Apr. 15, 2022.

(51) Int. Cl. C12N 15/81; C07K 14/395; C12N 9/00; C12N 9/80; C12P 3/00; C12R 1/865

(52) U.S. Cl. CPC C12N 15/81; C07K 14/395; C12N 9/80; C12N 9/93; C12P 3/00; C12R 2001/865; C12Y 305/01005; C12Y 603/04006

(57) ABSTRACT

The present invention provides for a genetically modified host cell comprising a first polypeptide capable of active transport of urea into the host cell and/or a second polypeptide capable of degrading urea into ammonia and carbon dioxide, wherein the genetically modified host cell is capable of degrading urea into ammonia and carbon dioxide. The genetically modified host cell in a medium comprising urea, a calcium salt or calcium ion, and a phosphate is capable of producing calcium phosphate.

### CROSS-REFERENCE TO RELATED APPLICATIONS

This application claims priority as a continuation application to PCT International Patent Application No. PCT/US2023/018715, filed Apr. 14, 2023, which claims priority to U.S. Provisional Patent Application Ser. No. 63/331,738, filed Apr. 15, 2022, which are both incorporated by reference in their entireties.

### STATEMENT OF GOVERNMENTAL SUPPORT

The invention described and claimed herein was made utilizing funds supplied by the U.S. Department of Energy under Contract No. DE-AC02-05CH11231. The government has certain rights in this invention.

### FIELD OF THE INVENTION

The present invention is in the field of producing calcium phosphate.

### BACKGROUND OF THE INVENTION

Currently there is no adequate biological production of a calcium phosphate.

### SUMMARY OF THE INVENTION

The present invention provides for a genetically modified host cell comprising a first polypeptide capable of active transport of urea into the host cell and/or a second polypeptide capable of degrading urea into ammonia and carbon dioxide, wherein the genetically modified host cell is capable of degrading urea into ammonia and carbon dioxide. The genetically modified host cell in a medium comprising urea, a calcium salt or calcium ion, and a phosphate is capable of producing calcium phosphate. In some embodiment, the calcium phosphate produced is amorphous calcium phosphate (ACP) particles and/or hydroxyapatite (HAP) crystals. In some embodiment, the ACP are particles intracellular ACP particles and the HAP crystals are extracellular platelet-like HAP crystals.

In some embodiments, the genetically modified host cell has an increased degradation urea into ammonia and carbon dioxide, wherein the rate of urea degradation of the genetically modified host cell is greater compared to the rate of urea degradation of an unmodified host cell. In some embodiments, the genetically modified host cell is genetically modified through DNA recombination, or through adaptive laboratory evolution, such as using urea as a sole nitrogen source. In some embodiments, the genetically modified host cell has a rate of degradation urea into ammonia and carbon dioxide that is equal or more than about any of the rates described herein.

In some embodiment, the genetically modified host cell is capable of producing nanoparticles. In some embodiment, the nanoparticles are ACP and/or HAP. In some embodiment, the nanoparticles are intracellular. In some embodiment, the nanoparticles are extracellular and/or crystalline. In some embodiment, the nanoparticles are essentially spherical or round. In some embodiment, the nanoparticles are essentially rods. In some embodiment, the nanoparticles have a diameter, or an average diameter, of, or at least, about 50 nm, 60 nm, 70 nm, 80 nm, 90 nm, 100 nm, 150 nm, 200 nm, 250 nm, or 300 nm, or any value within any two preceding values. In some embodiment, the rods have a length, or an average length, of, or at least, about 0.5 μm, 1.0 μm, 1.5 μm, 2.0 μm, 2.5 μm, 3.0 μm, 3.5 μm, 4.0 μm, 4.5 μm, or 5.0 μm, or any value within any two preceding values.

In some embodiment, when the genetically modified host cell is in a medium comprising a calcium salt or calcium ion, the genetically modified host cell the producing calcium phosphate.

In some embodiment, the first polypeptide capable of active transport of urea into the host cell is a urea active transporter. In some embodiment, the urea active transporter is a Saccharomyces boulardii or S. cerevisiae urea active transporter.

In some embodiment, the second polypeptide capable of degrading urea into ammonia and carbon dioxide is a urea amidolyase. In some embodiment, the urea amidolyase is a S. boulardii or S. cerevisiae urea amidolyase. In some embodiment, the nucleic acid encoding the urea amidolyase is operatively linked to a constitutive promoter. In some embodiment, the second polypeptide is a urease.

In some embodiment, the host cell comprises a urea active transporter and/or urea amidolyase native to the host cell, and the host cell comprises additional copy or copies of nucleic acid(s) encoding urea active transporter and/or urea amidolyase. In some embodiment, the nucleic acid(s) encoding urea active transporter and/or urea amidolyase are heterologous to the host cell.

In some embodiment, the genetically modified host cell comprises a first nucleic acid encoding the first polypeptide operatively linked to a first promoter capable of expressing the first polypeptide in the genetically modified host cell. In some embodiment, the genetically modified host cell comprises a second nucleic acid encoding the second polypeptide operatively linked to a second promoter capable of expressing the second polypeptide in the genetically modified host cell. In some embodiment, the first promoter and the second promoter are the same promoter.

In some embodiments, the additional copy or copies of nucleic acid(s) encoding urea active transporter and/or urea amidolyase are operatively linked to one or more promoters. In some embodiments, the one or more promoters are constitutive or inducible in the host cell. In some embodiment, the one or more promoters are heterologous to the host cell. In some embodiment, the one or more promoters are not subject to nitrogen regulation in the host cell. In some embodiment, when the host cell is a eukaryotic cell, the one or more promoters are constitutive promoters pTDH3 and/or pTEF1.

In some embodiment, the genetically modified host cell is modified to reduce or eliminate nitrogen catabolite repression with the host cell.

The present invention provides for a method for producing calcium phosphate, and/or a calcium phosphate biocomposite material, the method comprising: (a) providing a genetically modified host cell of the present invention in a medium comprising urea, a calcium salt or calcium ion, and a phosphate, and (c) culturing the genetically modified host cell to produce ammonia such that calcium phosphate is produced. In some embodiment, the calcium phosphate produced is intracellular amorphous calcium phosphate (ACP) particles and/or hydroxyapatite (HAP) crystals. In some embodiment, the ACP is intracellular ACP particles and the HAP crystals are extracellular platelet-like HAP crystals.

In some embodiments, the method further comprises shifting the pH of the medium. In some embodiments, the shifting comprises adding to the medium or mixing with the medium an acid to shift the pH down, or a base to shift the pH up. In some embodiments, the shifting comprises shifting the medium to a pH equal to more than about 7.0, 7.5, 8.0, 8.5, 9.0, 9.5, or 10.0, or within a range of any preceding two values. In some embodiments, the shifting results in the production of intracellular amorphous calcium phosphate (ACP) particles and/or hydroxyapatite (HAP) crystals. In some embodiments, the base is an inorganic base or an organic base. In some embodiments, the inorganic base is an alkali metal or alkali-earth metal hydroxide. In some embodiments, the alkali metal is Li, Na, K, Rb, or Cs. In some embodiments, the alkali-earth metal is Be, Mg, Ca, Sr, or Ba. In some embodiments, the base is ammonia.

Other objects, features, and advantages of the present invention will be apparent to one of skill in the art from the following detailed description and figures.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. Genomic sequences of S. boulardii engineered to hydrolyze urea. Constitutive expression of (A) dur3 integrated in the his3 auxotrophic region and (B) dur1/2 integrated in the trp1 auxotrophic region.

FIG. 2. Formation of intracellular and extracellular inorganic materials by engineered S. boulardii. (A) TEM image and (B) HAADF-STEM image of a small cluster of S. boulardii cells with intracellular ACP nanoparticles and extracellular platelet-like hydroxyapatite crystals. (C) STEM-EDS maps of (1) the region containing ACP nanoparticles and (2) the region containing platelet-like hydroxyapatite crystals both show Ca and P enrichment.

FIG. 3. ACP and platelet-like hydroxyapatite in genetically engineered S. boulardii. (A) TEM of a single S. boulardii cell containing ACP particles. Electron diffraction patterns show (B) ACP particles and (C) hydroxyapatite.

FIG. 4. Timeline of crystal formation in S. boulardii visualized by HAADF-STEM images of cell-crystal suspension: (A) After 15 hours growth cells formed small ACP particles. (B) Crystals appeared in solution after 15.5 hours growth resulting in hydroxyapatite covered cells and intracellular ACP particles. (C) After several days of growth hydroxyapatite flowers appeared.

FIG. 5. (A) In the presence of yeast cells, large clusters of crystalline materials were observed adjacent to the cells. Electron diffraction verified that these materials consist of hydroxyapatite. (B) Without yeast cells, large clusters of ACP nanoparticles were observed.

FIG. 6. (A) HAADF-STEM images of S. boulardii cell clusters. (B) STEM-EDS maps showed calcium signal enhancement in between S. boulardii cells.

FIG. 7. Osteoblast and osteoyeast.

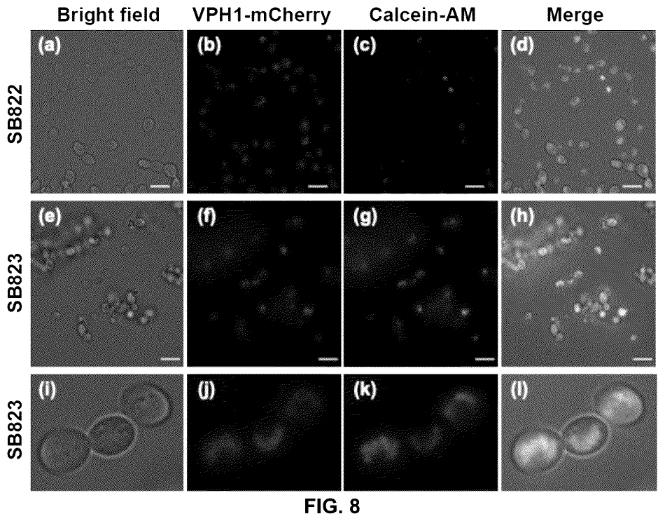

FIG. 8. Engineered yeast showing greater accumulation of calcium in the vacuoles. The images of yeast strains, SB822 (a-d) and SB823 (e-h), grown in the YNB media with 20 g/L urea, 50 mM Ca²⁺ and 10 uM calcein-AM are shown. We used 2 mg/mL Concanavalin A to minimize movement of these cells (see Methods). Three yeast cells located at the center of the panels (e-h) are magnified and shown (i-l). Bright field (a, e, i), channel for VPH1-mCherry (b, f, and j), channel for Ca²⁺-bound calcein (c, g, k), and merged (d, h, i) images are shown. The calcein channel contrast was set to the same minimum and maximum for each strain for comparison. The scale bar in each image represents 5 μm (e-l).

FIG. 9. HAp synthesis mediated by the osteoyeast platform analyzed using TEM. (a) The material produced by the osteoyeast platform collected using 10 um filter paper. (b) HAADF-STEM analysis of this material. STEM-EDX analyses of Ca (c) and P (d) of zone A and Ca (e) and P (f) of zone B. Electron diffraction analyses of zone A (g) and B (h). (i) XRD analysis of HAp synthesized by osteoyeast (light green) and bone (dark blue). The XRD spectra for bone is reproduced from the previous study (32).

FIG. 10. HAp synthesis mediated by the osteoyeast platform using correlative imaging analyses. (a) Scheme for correlative imaging analysis. (b-e) The time course of HAp synthesis mediated by the osteoyeast platform. The arrows indicate (a) emergence of extracellular vesicles, (b) fusion of two extracellular vesicles to a single vesicle, (c) conversion to HAp, and (d) exudate from the ruptured yeast cell. (f-h) Optical imaging of extracellular vesicles and HAP-like particles. The EVs and HAP-like particles appear in the mCherry channel (f), calcein (g) channel and are observable by bright-field imaging (h). (i-n) Analysis of a ruptured yeast cell during the HAp synthesis using fluorescent microscopy. (j) A HAADF-STEM analysis of a ruptured yeast cell. (k) A STEM-EDX analysis of Ca from the ruptured yeast cell. The asterisks on (i-k) denote the same locations analyzed using different microscopy methods. (l) Analysis of yeast cells during the HAp synthesis using fluorescent microscopy. (m,n) TEM analysis of (l) with an increased magnification. The asterisks on (l-n) denote the same locations analyzed using different microscopy methods. (o) A HAADF-STEM analysis of the zone denoted with the rectangle on. (p-r) STEM-EDX analyses of Ca, P, and C.

### DETAILED DESCRIPTION OF THE INVENTION

Before the invention is described in detail, it is to be understood that, unless otherwise indicated, this invention is not limited to particular sequences, expression vectors, enzymes, host cells, microorganisms, or processes, as such may vary. It is also to be understood that the terminology used herein is for purposes of describing particular embodiments only, and is not intended to be limiting.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” as used herein means a value that includes 10% less and 10% more than the value referred to.

The terms “host cell” and “host microorganism” are used interchangeably herein to refer to a living biological cell, such as a microorganism, that can be transformed via insertion of an expression vector. Thus, a host organism or cell as described herein may be a prokaryotic organism (e.g., an organism of the kingdom Eubacteria) or a eukaryotic cell. As will be appreciated by one of ordinary skill in the art, a prokaryotic cell lacks a membrane-bound nucleus, while a eukaryotic cell has a membrane-bound nucleus.

The term “heterologous DNA” as used herein refers to a polymer of nucleic acids wherein at least one of the following is true: (a) the sequence of nucleic acids is foreign to (i.e., not naturally found in) a given host cell; (b) the sequence may be naturally found in a given host cell, but in an unnatural (e.g., greater than expected) amount; or (c) the sequence of nucleic acids comprises two or more subsequences that are not found in the same relationship to each other in nature. The term “heterologous” as used herein refers to a structure or molecule wherein at least one of the following is true: (a) the structure or molecule is foreign to (i.e., not naturally found in) a given host cell; or (b) the structure or molecule may be naturally found in a given host cell, but in an unnatural (e.g., greater than expected) amount. For example, regarding instance (c), a heterologous nucleic acid sequence that is recombinantly produced will have two or more sequences from unrelated genes arranged to make a new functional nucleic acid. Specifically, the present invention describes the introduction of an expression vector into a host cell, wherein the expression vector contains a nucleic acid sequence coding for an enzyme that is not normally found in a host cell. With reference to the host cell's genome, then, the nucleic acid sequence that codes for the enzyme is heterologous.

The terms “expression vector” or “vector” refer to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also comprises materials to aid in achieving entry of the nucleic acid into the host cell, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host cell and replicated therein. Preferred expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The term “transduce” as used herein refers to the transfer of a sequence of nucleic acids into a host cell or cell. Only when the sequence of nucleic acids becomes stably replicated by the cell does the host cell or cell become “transformed.” As will be appreciated by those of ordinary skill in the art, “transformation” may take place either by incorporation of the sequence of nucleic acids into the cellular genome, i.e., chromosomal integration, or by extrachromosomal integration. In contrast, an expression vector, e.g., a virus, is “infective” when it transduces a host cell, replicates, and (without the benefit of any complementary virus or vector) spreads progeny expression vectors, e.g., viruses, of the same type as the original transducing expression vector to other microorganisms, wherein the progeny expression vectors possess the same ability to reproduce.

As used herein, the terms “nucleic acid sequence,” “sequence of nucleic acids,” and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing nonnucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog; intemucleotide modifications, such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., arninoalklyphosphoramidates, aminoalkylphosphotriesters); those containing pendant moieties, such as, for example, proteins (including nucleases, toxins, antibodies, signal peptides, poly-L-lysine, etc.); those with intercalators (e.g., acridine, psoralen, etc.); and those containing chelators (e.g., metals, radioactive metals, boron, oxidative metals, etc.). As used herein, the symbols for nucleotides and polynucleotides are those recommended by the IUPAC-IUB Commission of Biochemical Nomenclature (Biochem. 9:4022, 1970).

The term “operably linked” refers to a functional linkage between a nucleic acid expression control sequence (such as a promoter) and a second nucleic acid sequence, wherein the expression control sequence directs transcription of the nucleic acid corresponding to the second sequence.

In some embodiment, the first polypeptide capable of active transport of urea into the host cell is a urea active transporter, and comprises an amino acid sequence having equal to or more than 70%, 75%, 80%, 85%, 90%, 95%, or 99% identity to the amino acid sequence of a wild-type urea active transporter, such as S. boulardii or S. cerevisiae urea active transporter. In some embodiment, the first polypeptide comprises one or more, or all of the identical amino acid residues conserved between S. boulardii or S. cerevisiae urea active transporter.

In some embodiment, the second polypeptide capable of degrading urea into ammonia and carbon dioxide is a urea amidolyase, and comprises an amino acid sequence having equal to or more than 70%, 75%, 80%, 85%, 90%, 95%, or 99% identity to the amino acid sequence of a wild-type urea amidolyase, such as S. boulardii or S. cerevisiae urea amidolyase. In some embodiment, the second polypeptide comprises one or more, or all of the identical amino acid residues conserved between S. boulardii or S. cerevisiae urea amidolyase.

In some embodiment, the genetically modified host cell further comprises a nucleic acid encoding urease operatively linked to a promoter capable of expressing the urease in the genetically modified host cell. In some embodiment, the urease comprises an amino acid sequence having equal to or more than 70%, 75%, 80%, 85%, 90%, 95%, or 99% identity to the amino acid sequence of any wild-type urease.

In some embodiments, the genetically modified host cell is a genetically modified yeast (or osteoyeasts) capable of calcium phosphate and/or a calcium phosphate based composite biomaterial

In some embodiments, the genetically modified host cell is a yeast Saccharomyces cell, such as S. boulardii, engineered to constitutively hydrolyze urea. This yeast strain (“osteoyeast”) comprises one or more similar characteristics of how osteoblasts (bone cell) make bone. In the presence of Ca²⁺ ions, the yeast strain form internal vesicles carrying amorphous calcium phosphate. Additionally, hydroxyapatite is found on the outside of the cells. Calcium phosphate has a wide range of applications in biomedical areas, material sciences, water purification, and even fashion and apparel.

In some embodiments, a yeast Saccharomyces cell, such as S. boulardii, engineered to constitutively hydrolyze urea by leveraging 2 proteins, the urea transmembrane transporter DUR3 and urea amidolyase DUR1/2. Both dur3 and dur1/2 are endogenously present in yeasts like S. boulardii or S. cerevisiae to utilize urea as sole nitrogen source, but their expression is subject to nitrogen catabolite repression. In some embodiments, in order to overcome endogenous regulatory networks, one or more additional copies of dur3 and dur1/2 is integrated in the Saccharomyces cell chromosome, and constitutively expressed by the TDH3 and TEF1 promoter, respectively.

In some embodiments, the engineered yeast cells are grown in Yeast Nitrogen Media (YNB) with appropriate amino acid supplements, optionally supplemented with about 5 g/L ammonium sulfate. For crystal growths, about 5 g/L to 20 g/L urea and about 20 to 100 mM CaCl₂ is added to the media. Cells are grown for up to about 92 hours at about 30° C. to 37° C. Crystals/cells are harvested through a about 2.7 μm filter paper, washed with water, and left to dry at room temperature. In some embodiments, the cells or cell/crystal suspensions are immediately analyzed without filtering. The cells/crystals are imaged using transmission electron microscopy (TEM), high-angle annular dark-field scanning transmission electron microscopy (HAADF-STEM), and energy-dispersive X-ray spectroscopy (EDX), and the composition, phase and structure of the amorphous calcium phosphate and hydroxyapatite are identified.

In some embodiments, in the presence of urea and CaCl₂, the engineered yeast cells form internal vesicles composed of amorphous calcium phosphate. S. boulardii cells are also attached to external crystal structures, composed of crystalline calcium phosphate in the form of hydroxyapatite.

Calcium phosphate in the form of hydroxyapatite has a wide range of applications. Additionally, the system creates crystals that are physically attached to living yeast cells, giving us the unique advantage to functionalize cells for modification of biomaterial properties and applicability. For example, the yeast cells can be engineered to sense molecules in the environment, display proteins (for example antibodies) on its cell surface or form patterns through inducible biofilm formation.

The calcium phosphate based composite biomaterials produced by the present invention can be used in one or more of the following applications:
(A) Drug delivery system: Hydroxyapatite crystals are able to bind and store proteins, small molecules and DNA. At lower pH, the hydroxyapatite surface dissolves, enabling pH-dependent release of drugs.
(B) Biomedical applications: Calcium phosphate is the main component of teeth and bone (reviewed in Eliaz N. & Metoki N. Materials, 10, 334 (2017)). The platform enables the usage of the engineered yeast cells to function as producers for biomimetic teeth and bone precursors or coating.
(C) Water purification: Hydroxyapatite has been utilized to bind and remove contaminants from water. In addition, yeast cells can be engineered to bind and remove additional water contaminants or to function as sensors of water quality. The hydroxyapatite can be used in wastewater treatment, e.g., in the removal of urea. Examples include removal of fluoride (He J. et al J. Hazard. Mater. 303, 119-130 (2016)) or lead (Iconaru, S. I. et al Materials, 11, 2204 (2018)).
(D) Conductive and semiconductive materials: Calcium phosphate gel composites can be used as conductive material (Kasuga, T. et al. Adv. Mater. 14, 1490-1492 (2002)). In particular, composite materials (for example TiO2-hydroxyapatite) can be used as photocatalytic material, which has been utilized for NO removal (Yao J. et al. RSC Adv. 7, 24683 (2017)).
(E) Fashion and apparel.
(F) Light-weight hard materials: The dactyl club of mantis shrimp generates high forces and speeds, while being able to withstand those highspeed impacts. A recent publication investigating the composition and mechanisms showed that dactyl clubs contain layers of organic-inorganic composite materials of chitin and calcium phosphate, namely mineralized hydroxyapatite nanoparticles and amorphous carbonated calcium phosphate (Huang, W. et al. Nature Materials 19, 1236-1243 (2020)).
(G) Photonic properties: Recently, composite calcium phosphate glasses have been utilized for biophotonic applications due to their optical, biological, biocompatible and thermal properties (Pugliese, D. et al. ICTON 2016, Trento, Italy, We.C6.4).

In some embodiments, the genetically modified host cell can be functionalized for biosensing, production of protein drugs or metal sequestering.

In some embodiments, the nucleic acid encoding the first and/or second polypeptides are operatively linked to one or more promoters capable of transcription in the genetically modified host cell. In some embodiments, each nucleic acid of the one or more nucleic acids is a vector capable of stable introduction into and/or maintenance in the host cell.

In some embodiments, the genetically modified host cell is capable of producing one or more compounds in titers or yields equal to or more than the titers or yields described herein.

The nucleic acid constructs of the present invention comprise nucleic acid sequences encoding one or more of the subject polypeptide/enzymes. The nucleic acid of the subject polypeptide/enzymes is operably linked to promoters and optionally control sequences such that the subject enzymes are expressed in a host cell cultured under suitable conditions. The promoters and control sequences are specific for each host cell species. In some embodiments, expression vectors comprise the nucleic acid constructs. Methods for designing and making nucleic acid constructs and expression vectors are well known to those skilled in the art.

Sequences of nucleic acids encoding the subject enzymes are prepared by any suitable method known to those of ordinary skill in the art, including, for example, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature (e.g., in Matteuci et al. (1980) Tet. Lett. 521:719; U.S. Pat. Nos. 4,500,707; 5,436,327; and 5,700,637). In addition, the desired sequences may be isolated from natural sources by splitting DNA using appropriate restriction enzymes, separating the fragments using gel electrophoresis, and thereafter, recovering the desired nucleic acid sequence from the gel via techniques known to those of ordinary skill in the art, such as utilization of polymerase chain reactions (PCR; e.g., U.S. Pat. No. 4,683,195).

Each nucleic acid sequence encoding the desired subject enzyme can be incorporated into an expression vector. Incorporation of the individual nucleic acid sequences may be accomplished through known methods that include, for example, the use of restriction enzymes (such as BamHI, EcoRI, HhaI, Xhol, XmaI, and so forth) to cleave specific sites in the expression vector, e.g., plasmid. The restriction enzyme produces single stranded ends that may be annealed to a nucleic acid sequence having, or synthesized to have, a terminus with a sequence complementary to the ends of the cleaved expression vector. Annealing is performed using an appropriate enzyme, e.g., DNA ligase. As will be appreciated by those of ordinary skill in the art, both the expression vector and the desired nucleic acid sequence are often cleaved with the same restriction enzyme, thereby assuring that the ends of the expression vector and the ends of the nucleic acid sequence are complementary to each other. In addition, DNA linkers may be used to facilitate linking of nucleic acids sequences into an expression vector.

A series of individual nucleic acid sequences can also be combined by utilizing methods that are known to those having ordinary skill in the art (e.g., U.S. Pat. No. 4,683,195).

For example, each of the desired nucleic acid sequences can be initially generated in a separate PCR. Thereafter, specific primers are designed such that the ends of the PCR products contain complementary sequences. When the PCR products are mixed, denatured, and reannealed, the strands having the matching sequences at their 3′ ends overlap and can act as primers for each other Extension of this overlap by DNA polymerase produces a molecule in which the original sequences are “spliced” together. In this way, a series of individual nucleic acid sequences may be “spliced” together and subsequently transduced into a host cell simultaneously. Thus, expression of each of the plurality of nucleic acid sequences is effected.

Individual nucleic acid sequences, or “spliced” nucleic acid sequences, are then incorporated into an expression vector. The invention is not limited with respect to the process by which the nucleic acid sequence is incorporated into the expression vector. Those of ordinary skill in the art are familiar with the necessary steps for incorporating a nucleic acid sequence into an expression vector. A typical expression vector contains the desired nucleic acid sequence preceded by one or more regulatory regions, along with a ribosome binding site, e.g., a nucleotide sequence that is 3-9 nucleotides in length and located 3-11 nucleotides upstream of the initiation codon in E. coli. See Shine et al. (1975) Nature 254:34 and Steitz, in Biological Regulation and Development: Gene Expression (ed. R. F. Goldberger), vol. 1, p. 349, 1979, Plenum Publishing, N.Y.

Regulatory regions include, for example, those regions that contain a promoter and an operator. A promoter is operably linked to the desired nucleic acid sequence, thereby initiating transcription of the nucleic acid sequence via an RNA polymerase enzyme. An operator is a sequence of nucleic acids adjacent to the promoter, which contains a protein-binding domain where a repressor protein can bind. In the absence of a repressor protein, transcription initiates through the promoter. When present, the repressor protein specific to the protein-binding domain of the operator binds to the operator, thereby inhibiting transcription. In this way, control of transcription is accomplished, based upon the particular regulatory regions used and the presence or absence of the corresponding repressor protein. An example includes lactose promoters (LacI repressor protein changes conformation when contacted with lactose, thereby preventing the LacI repressor protein from binding to the operator). Another example is the tac promoter. (See deBoer et al. (1983) Proc. Natl. Acad. Sci. USA, 80: 21-25.) As will be appreciated by those of ordinary skill in the art, these and other expression vectors may be used in the present invention, and the invention is not limited in this respect.

Although any suitable expression vector may be used to incorporate the desired sequences, readily available expression vectors include, without limitation: plasmids, such as pSC101, pBR322, pBBR1MCS-3, pUR, pEX, pMR100, pCR4, pBAD24, pUC19; bacteriophages, such as M13 phage and λ phage. Of course, such expression vectors may only be suitable for particular host cells. One of ordinary skill in the art, however, can readily determine through routine experimentation whether any particular expression vector is suited for any given host cell. For example, the expression vector can be introduced into the host cell, which is then monitored for viability and expression of the sequences contained in the vector. In addition, reference may be made to the relevant texts and literature, which describe expression vectors and their suitability to any particular host cell.

The expression vectors of the invention must be introduced or transferred into the host cell. Such methods for transferring the expression vectors into host cells are well known to those of ordinary skill in the art. Those of ordinary skill in the art can transfect a host cell with a desired sequence using these or other methods.

For identifying a transfected host cell, a variety of methods are available. For example, a culture of potentially transfected host cells may be separated, using a suitable dilution, into individual cells and thereafter individually grown and tested for expression of the desired nucleic acid sequence. In addition, when plasmids are used, an often-used practice involves the selection of cells based upon antimicrobial resistance that has been conferred by genes intentionally contained within the expression vector, such as the amp, gpt, neo, and hyg genes.

When the host cell is transformed with at least one expression vector. When only a single expression vector is used (without the addition of an intermediate), the vector will contain all of the nucleic acid sequences necessary.

Once the host cell has been transformed with the expression vector, the host cell is allowed to grow. For microbial hosts, this process entails culturing the cells in a suitable medium. It is important that the culture medium contain an excess carbon source, such as a sugar (e.g., glucose) when an intermediate is not introduced. In this way, cellular production of the isoprenol ensured. When added, any intermediate is present in an excess amount in the culture medium.

The present invention provides for a method for constructing genetically modified yeast host cell of the present invention comprising: introducing one or more nucleic acid comprising open reading frames (ORF) encoding the polypeptide/enzyme described herein wherein each is operatively linked to a promoter capable of transcribing each ORF to which it is operatively linked.

### Host Cells

The genetically modified host cell can be any prokaryotic or eukaryotic cell, with any genetic modifications, capable of production of the olefinic ester in accordance with the methods of the invention. Suitable eukaryotic host cells include, but are not limited to, fungal cells. Suitable fungal cells are yeast cells, such as yeast cells of the Saccharomyces genus. Generally, although not necessarily, the host cell is a yeast or a bacterium. Any prokaryotic or eukaryotic host cell may be used in the present method so long as it remains viable after being transformed with a sequence of nucleic acids. In some embodiments, the host cell is not adversely affected by the transduction of the necessary nucleic acid sequences, the subsequent expression of the proteins (i.e., enzymes), or the resulting intermediates required for carrying out the steps associated with the mevalonate pathway. For example, it is preferred that minimal “cross-talk” (i.e., interference) occur between the host cell's own metabolic processes and those processes involved with the mevalonate pathway.

In some embodiments, the host cells are genetically modified in that heterologous nucleic acid have been introduced into the host cells, and as such the genetically modified host cells do not occur in nature. The suitable host cell is one capable of expressing a nucleic acid construct encoding one or more enzymes described herein. The gene(s) encoding the enzyme(s) may be heterologous to the host cell or the gene may be native to the host cell but is operatively linked to a heterologous promoter and one or more control regions which result in a higher expression of the gene in the host cell.

The enzyme can be native or heterologous to the host cell. Where the enzyme is native to the host cell, the host cell is genetically modified to modulate expression of the enzyme. This modification can involve the modification of the chromosomal gene encoding the enzyme in the host cell or a nucleic acid construct encoding the gene of the enzyme is introduced into the host cell. One of the effects of the modification is the expression of the enzyme is modulated in the host cell, such as the increased expression of the enzyme in the host cell as compared to the expression of the enzyme in an unmodified host cell.

Yeasts suitable for the invention include, but are not limited to, Yarrowia, Candida, Bebaromyces, Saccharomyces, Schizosaccharomyces and Pichia cells. In some embodiments, the yeast is Saccharomyces cerevisae. In some embodiments, the yeast is a species of Candida, including but not limited to C. tropicalis, C. maltosa, C. apicola, C. paratropicalis, C. albicans, C. cloacae, C. guillermondii, C. intermedia, C. lipolytica, C. panapsilosis and C. zeylenoides. In some embodiments, the yeast is Candida tropicalis. In some embodiments, the yeast is a non-oleaginous yeast. In some embodiments, the non-oleaginous yeast is a Saccharomyces species. In some embodiments, the Saccharomyces species is Saccharomyces boulardii or Saccharomyces cerevisiae. In some embodiments, the yeast is an oleaginous yeast. In some embodiments, the oleaginous yeast is a Rhodosporidium species. In some embodiments, the Rhodosporidium species is Rhodosporidium toruloides.

In some embodiments, the host cell is Rhodosporidium toruloides or Pseudomonas putida. In some embodiments, the host cell is a Gram negative bacterium. In some embodiments, the host cell is of the phylum Proteobactera. In some embodiments, the host cell is of the class Gammaproteobacteria. In some embodiments, the host cell is of the order Enterobacteriales. In some embodiments, the host cell is of the family Enterobacteriaceae. Examples of suitable bacteria include, without limitation, those species assigned to the Escherichia, Enterobacter, Azotobacter, Erwinia, Bacillus, Pseudomonas, Klebsielia, Proteus, Salmonella, Serratia, Shigella, Rhizobia, Vitreoscilla, and Paracoccus taxonomical classes.

Bacterial host cells suitable for the invention include, but are not limited to, Escherichia, Corynebacterium, Pseudomonas, Streptomyces, and Bacillus. In some embodiments, the Escherichia cell is an E. coli, E. albertii, E. fergusonii, E. hermanii, E. marmotae, or E. vulneris. In some embodiments, the Corynebacterium cell is Corynebacterium glutamicum, Corynebacterium kroppenstedtii, Corynebacterium alimapuense, Corynebacterium amycolatum, Corynebacterium diphtheriae, Corynebacterium efficiens, Corynebacterium jeikeium, Corynebacterium macginleyi, Corynebacterium matruchotii, Corynebacterium minutissimum, Corynebacterium renale, Corynebacterium striatum, Corynebacterium ulcerans, Corynebacterium urealyticum, or Corynebacterium uropygiale. In some embodiments, the Pseudomonas cell is a P. putida, P. aeruginosa, P. chlororaphis, P. fluorescens, P. pertucinogena, P. stutzeri, P. syringae, P. cremoricolorata, P. entomophila, P. fulva, P. monteilii, P. mosselii, P. oryzihabitans, P. parafluva, or P. plecoglossicida. In some embodiments, the Streptomyces cell is a S. coelicolor, S. lividans, S. venezuelae, S. ambofaciens, S. avermitilis, S. albus, or S. scabies. In some embodiments, the Bacillus cell is a B. subtilis, B. megaterium, B. licheniformis, B. anthracis, B. amyloliquefaciens, or B. pumilus.

### REFERENCES CITED HEREIN

1. U.N.D. of E.A.S. Affairs, United Nations Department of Economic and Social Affairs, The Sustainable Development Goals Report 2022. The Sustainable Development Goals Report (2022).
2. Net Zero Emissions by 2050 Scenario (NZE)-World Energy Model-Analysis. IEA, (available at webpage at: iea.org/reports/world-energy-model/net-zero-emissions-by-2050-scenario-nze).
3. IEA—International Energy Agency. IEA, (available at webpage at: iea.org/).
4. Y.-K. Son, Y.-L. Zhao, D.-C. Ko, B.-M. Kim, Prediction of cracking initiation in enamel coated product and its verification by four-point bending test. J. Mech. Sci. Technol. 28, 1481-1489 (2014).
5. B. N. A. Al-Gabri, A. B. Nabilah, F. N. Abdul Aziz, I. A. Karim, Numerical analysis of out-of-plane deformation of shear wall. IOP Conf. Ser.: Earth Environ. Sci. 357, 012001 (2019).
6. B. Mostefa, A. Abdelkrim, B. Ali, B. Mohamed, Effect of hardening induced by cold expansion on damage fatigue accumulation and life assessment of Aluminum alloy 6082 T6. Mater. Res. 15, 981-985 (2012).
7. W. Huang, M. Shishehbor, N. Guarin-Zapata, N. D. Kirchhofer, J. Li, L. Cruz, T. Wang, S. Bhowmick, D. Stauffer, P. Manimunda, K. N. Bozhilov, R. Caldwell, P. Zavattieri, D. Kisailus, A natural impact-resistant bicontinuous composite nanoparticle coating. Nat. Mater. 19, 1236-1243 (2020).
8. K. Okuda, K. Hirota, T. Mizutani, Y. Numamoto, Enhanced toughness of hydroxyapatite-poly (ethylene terephthalate) composites by immersion in water. Materials Advances. 2, 5691-5703 (2021).
9. R. C. Thomson, M. J. Yaszemski, J. M. Powers, A. G. Mikos, Hydroxyapatite fiber reinforced poly(alpha-hydroxy ester) foams for bone regeneration. Biomaterials. 19, 1935-1943 (1998).
10. N. Ranjan, R. Singh, I. P. S. Ahuja, Investigations for mechanical properties of PLA-HAp-CS based functional prototypes. Materials Today: Proceedings. 18, 2329-2334 (2019).
11. Hydroxyapatite market. Allied Market Research, (available at webpage at: alliedmarketresearch.com/hydroxyapatite-market).
12. G. Ma, Three common preparation methods of hydroxyapatite. IOP Conf. Ser.: Mater. Sci. Eng. 688, 033057 (2019).
13. T. Czigler, S. Reiter, P. Schulze, K. Somers, Laying the foundation for zero-carbon cement (2020), (available at webpage at: mckinsey.com/industries/chemicals/our-insights/laying-the-foundation-for-zero-carbon-cement).
14. S. Boonrungsiman, E. Gentleman, R. Carzaniga, N. D. Evans, D. W. McComb, A. E. Porter, M. M. Stevens, The role of intracellular calcium phosphate in osteoblast-mediated bone apatite formation. Proc. Natl. Acad. Sci. U.S.A. 109, 14170-14175 (2012).
15. A. Lotsari, A. K. Rajasekharan, M. Halvarsson, M. Andersson, Transformation of amorphous calcium phosphate to bone-like apatite. Nat. Commun. 9, 4170 (2018).
16. T. Iwayama, T. Okada, T. Ueda, K. Tomita, S. Matsumoto, M. Takedachi, S. Wakisaka, T. Noda, T. Ogura, T. Okano, P. Fratzl, T. Ogura, S. Murakami, Osteoblastic lysosome plays a central role in mineralization. Sci Adv. 5, eaax0672 (2019).
17. A. Ballabio, J. S. Bonifacino, Lysosomes as dynamic regulators of cell and organismal homeostasis. Nat. Rev. Mol. Cell Biol. 21, 101-118 (2020).
18. K. M. Holmström, N. Marina, A. Y. Baev, N. W. Wood, A. V. Gourine, A. Y. Abramov, Signalling properties of inorganic polyphosphate in the mammalian brain. Nat. Commun. 4, 1362 (2013).
19. K. Chuprunov, A. Yudin, D. Lysov, E. Kolesnikov, D. Kuznetsov, D. Leybo, I. Ilinykh, A. Godymchuk, The pH Level Influence on Hydroxyapatite Phase Composition Synthesized with Hydrothermal Method. IOP Conf. Ser.: Mater. Sci. Eng. 731, 012023 (2020).
20. M. N. Hossain, S. Afrin, S. Humayun, M. M. Ahmed, B. K. Saha, Identification and Growth Characterization of a Novel Strain of Saccharomyces boulardii Isolated From Soya Paste. Front Nutr. 7, 27 (2020).
21. S. C. Li, P. M. Kane, The yeast lysosome-like vacuole: endpoint and crossroads. Biochim. Biophys. Acta. 1793, 650-663 (2009).
22. M. Hothorn, H. Neumann, E. D. Lenherr, M. Wehner, V. Rybin, P. O. Hassa, A. Uttenweiler, M. Reinhardt, A. Schmidt, J. Seiler, A. G. Ladurner, C. Herrmann, K. Scheffzek, A. Mayer, Catalytic core of a membrane-associated eukaryotic polyphosphate polymerase. Science. 324, 513-516 (2009).
23. K. Papouskova, L. Jiang, H. Sychrova, Vcx1 and ESCRT components regulate intracellular pH homeostasis in the response of yeast cells to calcium stress. FEMS Yeast Res. 15 (2015).
24. K. Zhao, M. Bleackley, D. Chisanga, L. Gangoda, P. Fonseka, M. Liem, H. Kalra, H. Al Saffar, S. Keerthikumar, C.-S. Ang, C. G. Adda, L. Jiang, K. Yap, I. K. Poon, P. Lock, V. Bulone, M. Anderson, S. Mathivanan, Extracellular vesicles secreted by Saccharomyces cerevisiae are involved in cell wall remodelling. Commun Biol. 2, 305 (2019).
25. J. Rizzo, A. Taheraly, G. Janbon, Structure, composition and biological properties of fungal extracellular vesicles. microLife. 2 (2021).
26. A. Sethuraman, N. N. Rao, A. Kornberg, The endopolyphosphatase gene: essential in Saccharomyces cerevisiae. Proc. Natl. Acad. Sci. U.S.A. 98, 8542-8547 (2001).
27. R. Gerasimaitė, A. Mayer, Ppn2, a novel Zn2+-dependent polyphosphatase in the acidocalcisome-like yeast vacuole. J. Cell Sci. 130, 1625-1636 (2017).
28. J. Zhu, Z.-T. Zhang, S.-W. Tang, B.-S. Zhao, H. Li, J.-Z. Song, D. Li, Z. Xie, A Validated Set of Fluorescent-Protein-Based Markers for Major Organelles in Yeast (Saccharomyces cerevisiae). MBio. 10 (2019).
29. S. J. Du, V. Frenkel, G. Kindschi, Y. Zohar, Visualizing normal and defective bone development in zebrafish embryos using the fluorescent chromophore calcein. Dev. Biol. 238, 239-246 (2001).
30. D. Halachmi, Y. Eilam, Cytosolic and vacuolar Ca2+ concentrations in yeast cells measured with the Ca2+-sensitive fluorescence dye indo-1. FEBS Lett. 256, 55-61 (1989).
31. T. Dunn, K. Gable, T. Beeler, Regulation of cellular Ca2+ by yeast vacuoles. J. Biol. Chem. 269, 7273-7278 (1994).
32. E. Davies, K. H. Müller, W. C. Wong, C. J. Pickard, D. G. Reid, J. N. Skepper, M. J. Duer, Citrate bridges between mineral platelets in bone. Proc. Natl. Acad. Sci. U.S.A. 111,E1354-63 (2014).
33. E. S. Thian, S. M. Best, “18—Si-substituted hydroxyapatite” in Bioceramics and their Clinical Applications, T. Kokubo, Ed. (Woodhead Publishing, 2008), pp. 424-437.
34. A. Ressler, A. Žužić, I. Ivanišević, N. Kamboj, H. Ivanković, Ionic substituted hydroxyapatite for bone regeneration applications: A review. Open Ceramics. 6, 100122 (2021).
35. H. S. Vilela, M. C. Rodrigues, B. M. Fronza, R. B. Trinca, F. M. Vichi, R. R. Braga, Effect of temperature and pH on calcium phosphate precipitation. Cryst. Res. Technol. 56, 2100094 (2021).

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Engineering Osteoyeast for the Production of Calcium Phosphate Based Composite Biomaterials

(1) Strategy to Engineer Saccharomyces boulardii

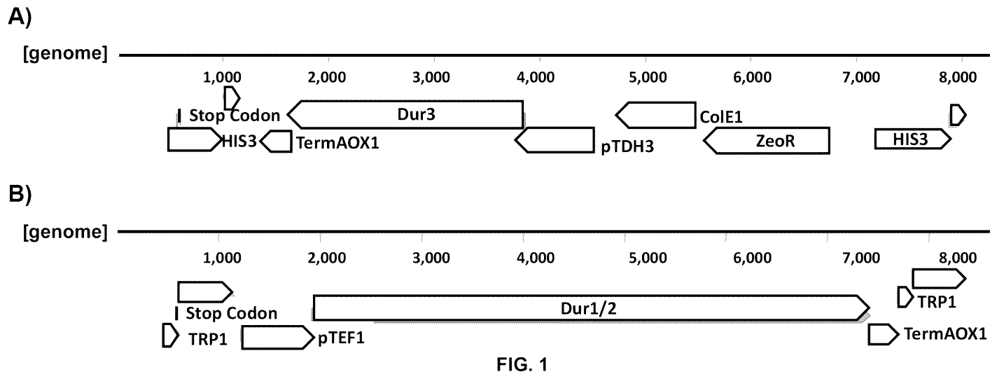

For urea hydrolysis in yeast S. boulardii, gene expression of two genes is required: Urea active transporter (dur3), required for active transport of urea into the cell, and urea amidolyase (dur1/2), required for degradation of urea in a two step reaction to ammonia and carbon dioxide. Both genes are endogenously present in S. boulardii, but are subject to nitrogen catabolite repression (Zhao et al. Nitrogen regulation involved in the accumulation of urea in Saccharomyces cerevisiae. Yeast, 30 (2013), 437-447.) For that reason, we inserted an additional copy of dur3 and dur1/2 controlled by constitutive promoters (pTDH3 and pTEF1) into the his3 and trp1 auxotrophic markers in S. boulardii genome (FIG. 1)

(2) Inorganic Materials Formation in S. boulardii

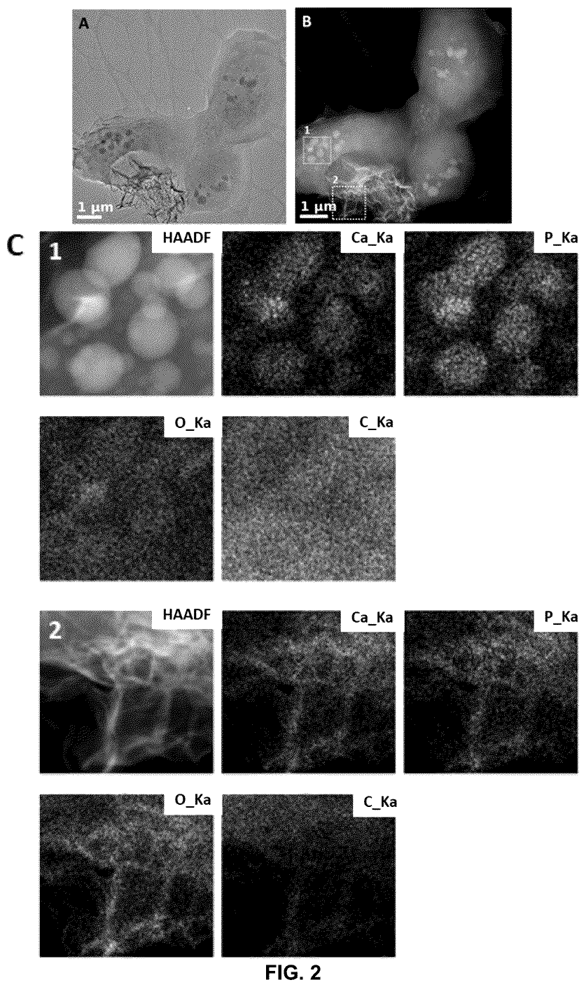

Inorganic materials produced through urea hydrolysis in S. boulardii formed within 15 hours of growth at 37° C. in the presence of urea and CaCl₂. Transmission electron microscopy (TEM) and high-angle annular dark-field scanning transmission electron microscopy (HAADF-STEM) showed that the inorganic materials consisted of two different morphologies, either as roughly round nanoparticles with diameters of 50-300 nm located in the periphery of the S. boulardii cells or as clusters of extracellular crystal rods that span several micrometers (FIG. 2). HAADF-STEM energy-dispersive X-ray spectroscopy (EDS) measurements confirmed that both forms of inorganic materials were composed of calcium phosphate. The regions with higher mass density, which appear bright in HAADF-STEM images, showed a high intensity of Ca and P elements that demonstrated the formation of calcium phosphate (FIG. 2, panel C).

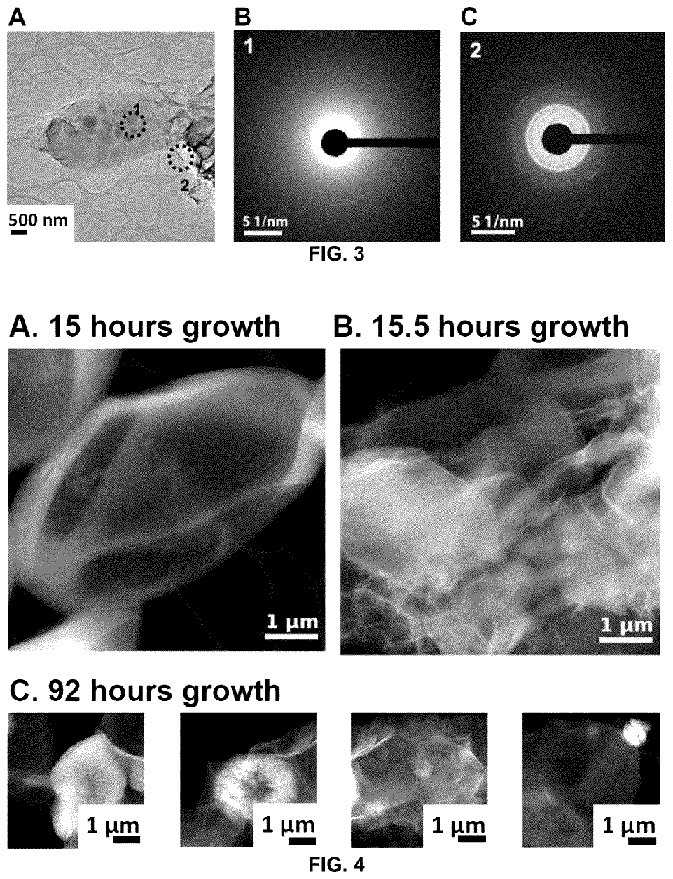

Electron diffraction patterns showed two calcium phosphate crystal morphologies: intracellular amorphous calcium phosphate (ACP) particles and extracellular platelet-like hydroxyapatite (HAP) crystals (FIG. 3). The diffraction pattern taken from the region containing intracellular nanoparticles within the cell showed no crystalline diffraction rings, which are characteristic of polycrystalline structures (FIG. 3, panel B). In contrast, the extracellular hydroxyapatite showed sharp diffraction rings matching the interplanar spacings of crystalline hydroxyapatite (FIG. 3, panel C).

(3) Time Course of Crystal Formation in S. boulardii

In preliminary studies we found that the pH of the growth media rose to >5.8 due to urea hydrolysis. Even though a more exact timeline and pH threshold needs to be established, calcium phosphate inorganic materials formed rapidly within minutes at this pH, resulting in a pH drop to around 5.2, followed by pH rising again in the next hours due to ongoing urea hydrolysis.

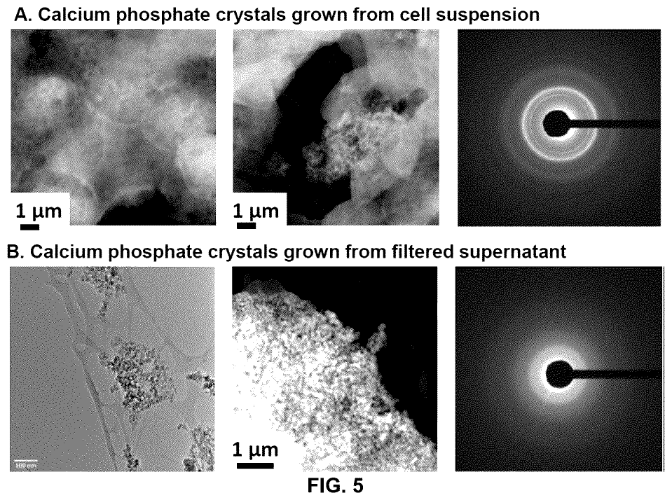

TEM analysis revealed that intracellular ACP particle formation occurred before external HAP crystals were visible (FIG. 4, panel A). As calcium phosphate composites became visible in culture 30 minutes later, cells were both covered by hydroxyapatite and contained intracellular ACP particles (FIG. 4, panel B). Longer incubation (i.e. days) resulted in the formation of flower-like HAP crystals (FIG. 4, panel C).

(4) Mechanism of Crystal Formation in S. boulardii

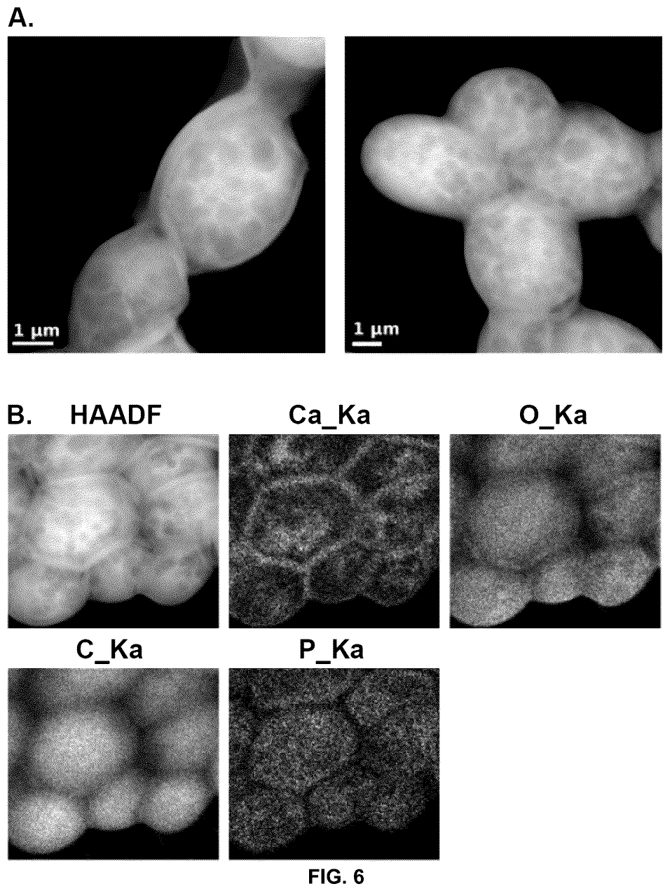

In order to investigate if living S. boulardii cells are required for the formation of hydroxyapatite external to the cells, wild type S. boulardii cells were grown in media containing urea and CaCl₂. As expected, the cells were not able to hydrolyze urea and no external crystals formed. After adjusting the pH to around 7 by addition of NaOH, inorganic materials were immediately visible in culture. TEM analysis showed hydroxyapatite formation covering the yeast cells (FIG. 5, panel A). On the other hand, engineered S. boulardii cells were grown in the presence of urea, but no CaCl₂. As expected, the pH rose but no external crystals formed. Yeast cells were removed from this high pH culture using a 0.2 μm filter. Addition of 50 mM CaCl₂ to the filtered supernatant resulted in the formation of amorphous calcium phosphate, not hydroxyapatite (FIG. 5, panel B). To sum up, living S. boulardii cells are required for the formation of hydroxyapatite.

Cell flocculation is a well known phenomenon in yeast fermentation. While the underlying mechanisms are still under investigation, a recent publication suggests that calcium plays a major role in yeast flocculation at high pH (Rogowska A. et al, The influence of different pH on the electrophoretic behavior of Saccharomyces cerevisiae modified by calcium ions. Sci. Rep., 8 (2018), 7261). As the pH of the media increases, functional groups of the yeast cell wall (e.g. phosphate and carboxyl groups) get deprotonated, resulting in a negative charge. Calcium ions form cationic bridges between yeast cells resulting in yeast flocculation.

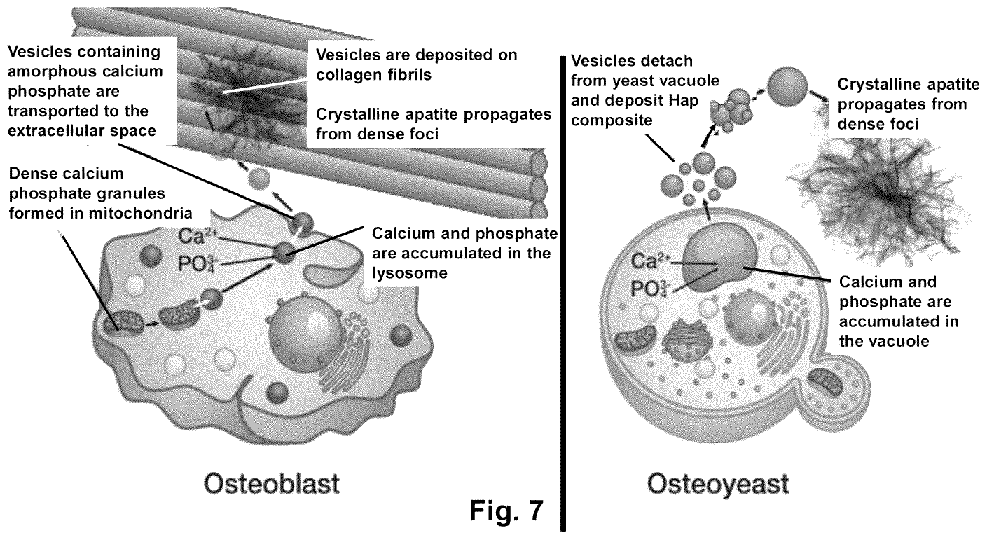

This phenomenon was observed when engineered S. boulardii was grown in phosphate limited media in the presence of urea and CaCl₂. Cell clumps formed without hydroxyapatite or ACP formation (FIG. 6, panel A). Elemental mapping revealed calcium concentration enhancement in between the S. boulardii cells (FIG. 6, panel B). The absence of strong phosphate signals is a result of utilization of polyphosphate storage due to absence of external phosphate.

### (A) Hypothesis 1: Extracellular HAP Formation

External hydroxyapatite formation seems to be triggered by pH increase in the media (at around pH of 6). We hypothesize that calcium is concentrated near the S. boulardii cell surfaces, as functional groups are deprotonated at increasing pH resulting in a negative charge. Once the critical pH for calcium phosphate formation is reached, calcium phosphate should preferably form and precipitate near the S. boulardii cell surface, as local calcium concentrations are high there. Organic components of the S. boulardii cell wall could favor the formation of hydroxyapatite, rather than other calcium phosphate crystal morphologies (like amorphous calcium phosphate or dicalcium phosphate dihydrate).

### (B) Hypothesis 2: Intracellular ACP Particle Formation

Urea hydrolysis, occurring in the S. boulardii cytoplasm, leads to an intracellular pH increase. Consequently, protons could be pumped from the vacuole into the cytoplasm, for example through the vacuolar calcium ion transporter Vcx1, which would result in an increased calcium concentration inside the vacuole. Vacuoles in S. boulardii are also the main storage sites for polyphosphate. Once the critical pH for calcium phosphate precipitation is reached inside the vacuole (due to protons being pumped out), ACP particles could be formed through the increased abundance of calcium inside the vacuole and polyphosphate storage. This pH increase is most likely reached earlier than the pH increase of the surrounding media and therefore intracellular ACP particles are observed earlier than external hydroxyapatite formation.

### (C) Future Work

The formation and progression of ACP particles can be investigated by live imaging through super resolution microscopy. One can use live cell stains to visualize components inside and around the cells, in particular calcein-AM to stain intracellular calcium (for ACP particles), calcein to stain extracellular calcium (for HAP crystals) or DAPI to stain the nucleus and vacuolar polyphosphate vesicles. One can also utilize fluorescent proteins fused to endogenously expressed organelle specific proteins to be able to identify ACP particle location, formation and potential organelle trafficking/secretion. The ultimate goal is to produce correlative live cell imaging with TEM endpoint reads.

### (6) Materials and Methods

The Saccharomyces boulardii strain with auxotrophic mutants (trip1, his3, ura3) was provided by the Jin lab (Liu, J. J. et al Metabolic Engineering of Probiotic Saccharomyces boulardii. Applied and Environmental Microbiology, 82 (2016), 2280-2287). Yeast transformations were carried out using the LiAc method. Engineered yeast cells were grown in Yeast Nitrogen Media (YNB) with appropriate amino acid supplements, optionally supplemented either at the beginning or at a later time point with 20 g/L urea and/or 50 mM CaCl₂. Cells were grown for up to 92 hours at 37° C. Crystals/cells were harvested through a 2.7 μm filter paper, washed with water and left to dry at room temperature. In some instances, cells or cell/crystal suspensions were immediately analyzed without filtering.

Dry pellets containing yeast cells and inorganic materials were dispersed in distilled water and deposited onto lacey carbon 300 mesh copper grids. TEM imaging and selected-area electron diffraction (SAED) were performed at the National Center for Electron Microscopy on a FEI ThemIS TEM equipped with a X-FEG gun and a Ceta2 complementary metal oxide semiconductor (CMOS) camera operating at 300 kV. High-angle annular dark field (HAADF) images were acquired in scanning transmission electron microscopy (STEM) mode at 300 kV with a convergence semi-angle of 11.3 mrad. To determine the compositions of the inorganic materials, energy dispersive X-ray spectroscopy (EDS) was performed using a Bruker SuperX windowless EDS detector, which has a solid angle of 0.7 steradian enabling high count rates with minimal dead time for fast STEM-EDS mapping. STEM-EDS elemental mapping was performed at 300 kV with a 5 min acquisition time.

### Example 2

### A Synthetic Osteoyeast Platform for Production of Bone-Like Hydroxyapatite

Hydroxyapatite (HAp) is a major component of biocomposites such as bone and teeth. Strong and tough yet lightweight and biodegradable, HAp-based composites have many clinical applications and have great potential to reduce reliance on carbon-intensive commodity materials such as plastics, building materials, and aluminum. However, the current chemical HAp synthesis has high cost and environmental footprints. Herein is demonstrated a synthetic osteoyeast platform for HAp biosynthesis, designed leveraging the mechanism osteoblasts use. Correlative imaging analyses reveals that expression of ureolytic enzymes enable yeast vacuoles to function as devices to transport, accumulate, and store amorphous calcium phosphate (ACP). This yeast strain subsequently secretes ACP in extracellular vesicles, and it is transformed to HAp, suggesting that synthesis of HAp is not a unique ability of higher eukaryotes. Instead, it might have evolved from the calcium detoxification of their common ancestors. Further experimentation suggests that this platform could produce HAp from dilute sources of calcium phosphate, which would allow diverse cost-effective and abundant feedstocks such as wastewater to be tapped for sustainable production of HAp and HAp composites.

### Introduction

Climate change associated with increasing greenhouse gas (GHG) emissions from human activities is one of the major challenges faced by people around the globe. The United Nations states that GHG emissions must reach net zero by 2050 to limit the increase of global average temperature to 1.5° C. and avoid the worst effects of climate change (1). Manufacture of commodity materials such as plastics and building materials accounts for over 10% of GHG emissions (2, 3).

Hydroxyapatite (HAp) composites may be attractive next-generation alternatives to all these materials. For example, HAp is highly compatible with both synthetic and biological polymers and can form diverse composite materials that are generally lightweight but have exceptionally high strength, toughness, and impact resistance (4-6)⁽⁷⁾. Bones and teeth (composites of HAp and proteins) are classic examples of HAp composites. Their mechanical properties are comparable to those of building materials. Additionally, HAp can be infused during manufacturing into plastics such as polyethylene telephthalate (PET) (8), nyron and bioplastics (9)^{,(10)} to reinforce them, reducing plastic content of each item and thereby our reliance on fossil fuels.

The HAp market is already significant, estimated to reach USD 3.65 billion globally by 2027 (11). HAp is currently produced mainly for orthopedic and dental applications, typically using hydrothermal, wet precipitation, or sol-gel methods (12). However, HAp synthesis is generally energy intensive, with a large environmental footprint. For example, the hydrothermal production method uses limestone (CaCO₃) as a feedstock. The limestone is first calcined at a high temperature (>900° C.) to split CO₂, yielding CaO, which then is reacted with phosphate at high temperature and pressure. The wet precipitation relies on chemical reactions between calcium and phosphorus ions under a controlled pH and temperature of the solution. The precipitated powder is typically calcined at 400-600° C. or as much as 1,200° C. to obtain HAp with desired characteristics. While we were unable to find a life-cycle assessment (LCA) for current HAp synthesis processes, more than 1 kg CO₂ is likely emitted for every kilogram of HAp produced using the hydrothermal method, based on the LCA for cement production (13).

Synthetic biology may offer a greener route for HAp and HAp composite synthesis. For example, osteoblasts, which are specialized cells for bone formation (and thereby for HAp synthesis) can synthesize HAp at mild temperatures. Because of their abilities to transport, accumulate, and store specific ions, osteoblasts can synthesize HAp from calcium and phosphate ions in the dilute sources such as the bloodstream (which contains 2.0-2.5 mM Ca and 1.1-1.5 mM PO₄). This ability infers that we may be able to make HAp from diverse feedstocks using biological systems. However, osteoblasts are not amenable to large-scale fermentation, making pursuit of HAp production from osteoblasts unattractive. Therefore, a strategy is sought for engineering yeast as an alternative platform for HAp synthesis, since yeasts are the industrial standard for fermentative production of commodity chemicals.

Herein is presented a successful engineering of a yeast platform for production of HAp using the known mechanisms for HAp synthesis in osteoblasts as a design principle. Correlative optical and electron microscopy imaging suggests that this engineered osteoblast-like yeast (osteoyeast) platform indeed uses mechanisms similar to those osteoblasts use to make HAp. These results suggest that the ability of osteoblasts to synthesize HAp is not unique to higher eukaryotes and may have evolved from calcium detoxification in common ancestral eukaryotes. Further experiments confirm that this osteoyeast platform can efficiently synthesize HAp from media containing low concentrations of calcium and phosphate (˜1 mM). High yield is reached, suggesting that this approach can enable HAp synthesis from diverse feedstocks (e.g., phosphorite, slag, waste water). This example brings us closer to being able to use osteoyeasts for HAp synthesis, lowering both the carbon footprints and the costs of HAp and HAp composite manufacture.

### Results

### A Design Principle for Engineering an Osteoyeast Platform

A known mechanism for HAp synthesis in osteoblasts is used as a model to design and build an osteoyeast strain. The HAp synthesis process in osteoblasts proceeds in four steps (14-16). In Step 1, osteoblasts use lysosomes to transport, accumulate, and store phosphate and calcium. This organelle, an acidic compartment important for organismal homeostasis (17), contains polyphosphate (18). Both low pH and the presence of polyphosphate may be important for maintaining an amorphous calcium phosphate (ACP) phase in this organelle. In Step 2, as the lysosomes are filled with ACP, they are secreted into the extracellular milieu as matrix vesicles (MVs), in which polyphosphate is gradually replaced with monophosphate. In Step 3, these MVs interact with proteins such as collagen and are gradually deformed, releasing ACP. In Step 4, platelet-like HAp is formed within the gap of collagen fibrils, using them as templates. These four steps are used as a basis for developing a design principle we could follow when engineering our osteoyeast platform.

As a chassis for HAp synthesis, we selected Saccharomyces boulardii rather than the standard model yeast, S. cerevisiae. For chemical HAp synthesis, it is important to maintain high pH, typically from 7 to 11 (19). As a probiotic strain, S. boulardii can tolerate a wider range of media pH than S. cerevisiae (20). The yeast vacuole, a lysosome-like organelle, is selected as an engineering target. This acidic compartment in the yeast cell plays an important role in pH and ion homeostasis, storage of ions as well as tolerance of toxic metals (21). The vacuole is filled with polyphosphate, which is synthesized via the VTC complex using ATP as a substrate (22). An H⁺-antiporter, VCX1 (H⁺ antiporter), plays an important role in transporting calcium into the vacuole and in controlling intracellular pH (23). It is therefore hypothesized that increasing cytosolic pH activates the vacuoles to transport, accumulate, and store calcium as a form of ACP. The cytosolic pH can be augmented by urea decomposition through expression of ureolytic enzymes such as urea amydolyase.

The molecular mechanisms underlying secretion of MV, filled with ACP, in the osteoblasts have yet to be characterized and implemented in the yeasts. However, Smith and Jones (2021) reported that yeast can also produce extracellular vesicles (EVs) and that the EVs were likely associated with vacuoles (24, 25). This process may be used to engineer osteoyeasts to secrete ACP as EVs. Additionally, in Step 2 of the HAp synthesis process in osteoblasts, polyphosphate in the EVs must be replaced with monophosphate. For this, Ppn1/2 are known to degrade polyphosphate into monophosphate (26, 27). The polyphosphate synthesis and decomposition catalyzed by the VTC complex and Ppn1/2, respectively, are carefully balanced to maintain homeostasis in the yeast cells. However, once the EVs are secreted, ATP is not available for synthesis of polyphosphate, and polyphosphate is likely degraded to monophosphate. Conversion of ACP into HAp in Steps 3 and 4 is mediated through abiotic processes and can occur as long as a suitable environment is provided.

### Expression of Ureolytic Enzymes Enabled Yeast Cells to Accumulate Calcium in Vacuoles

Following this design principle for developing the osteoyeast platform, the S. boulardii strain (SB818) is first engineered to express genes for ureolytic enzymes, urea amidolyase (dur12) and urea transporter (dur3) (SB819-823). A list of the engineered S. boulardii strains is summarized in Table 1. The urease activity of SB818-820 (Supplementary FIG. 1) is subsequently tested; only SB820 showed the increased urease activity and culture pH, suggesting that both dur12 and dur3 are required for urea decomposition. These results suggests that SB820 could increase the intracellular pH.

To confirm whether the engineered strain can accumulate calcium in vacuoles, we investigated cellular processes during urea decomposition. To visualize the vacuole membrane, we expressed a gene coding V-type proton ATPase subunit A (Vph1) fused with a gene coding an mCherry fluorescent protein (mCherry) and created SB821-823. We adapted this technique, which has been validated in S. cerevisiae (28). In addition, we used calcein-acetoxymethyl ester (AM) to monitor accumulation of calcium. Calcein-AM is a non-fluorescent molecule, but when it is taken up in cells, it is hydrolyzed by intracellular esterase to calcein, which is fluorescent upon binding to calcium ions (29).

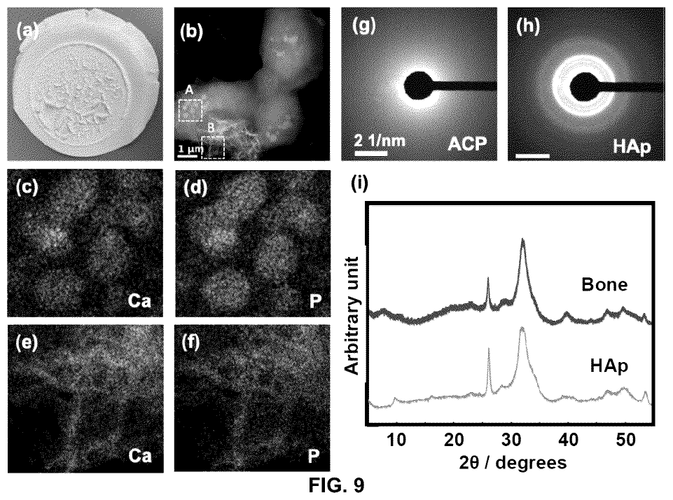

The SB822 and SB823 were grown in the modified YNB media in a glass bottom petri dish coated with Concanavalin A. The ureolytic reaction was initiated by addition of 20 g/L urea and 10 μM calcein-AM. An analysis using fluorescent microscopy indicated that the calcein signal was accumulated in vacuoles of both strains (FIG. 8). In SB822, the calcein signals increased within the vacuoles over 4 hours (FIG. 8). This result is in line with a previous work showing that vacuoles store ions, including magnesium and calcium (30, 31). Calcein signals were intensified in the SB823 vacuoles (FIG. 8), indicating that SB823 can accumulate more calcium in the vacuole. Additionally, SB823 increased the culture pH (pH 6.06), but SB822 did not (pH 3.97). These results indicated that we successfully engineered vacuoles as devices to transport, accumulate, and store Ca²⁺. Furthermore, crystal-like substances appeared only in the SB823 cultures (FIG. 8), suggesting that SB823 was involved in the formation of these substances.

TABLE 1
S. boulardii strains used.

| Strain names | Modifications |
|---|---|
| SB818 | wild type S. boulardii |
| SB819 | pTDH3-dur12(urea amidolyase) |
| SB820 | pTDH3-dur12, pTEF-dur3(urea transporter) |
| | (dual plasmid system) |
| SB821 | pTDH3-dur12, pTEF-dur3 (single plasmid system) |
| SB822 | Vph1-mCherry (V-type proton ATPase subunit A with |
| | a gene coding an mCherry fluorescent protein) |
| SB823 | pTDH3-dur12, pTEF-dur3 (single plasmid |
| | system), Vph1-mCherry |

### Transmission Electron Microscopy Analysis Suggested the Engineered Yeast Can Mediate HAp Synthesis

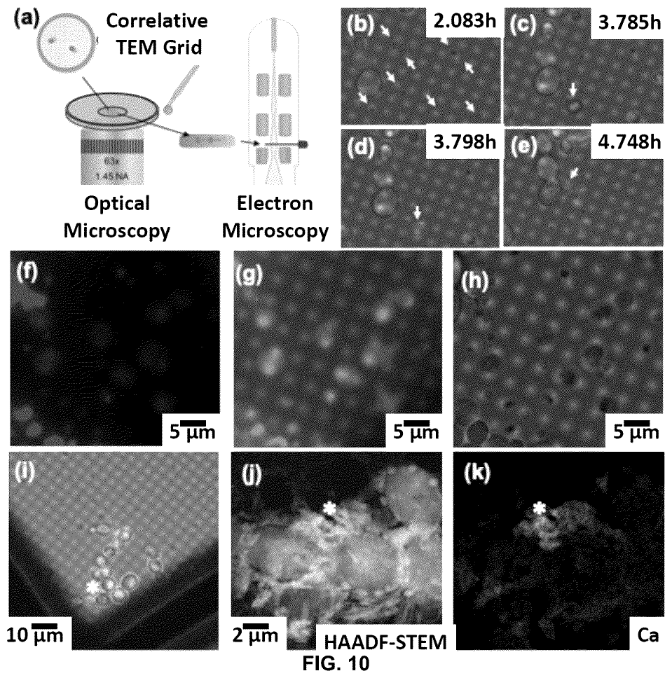

To further characterize the vacuolar content and extracellular crystal-like substances, the SB820 culture is analyzed using a high-angle annular dark-field scanning transmission electron microscope (HAADF-STEM). After cultivation of SB820 in the modified YNB media for less than 24 hours, white precipitants are visible and removed (FIG. 9). This material is washed with deionized water, dried, and analyzed (FIG. 9). This analysis reveals that the precipitants are mixtures of cells and calcium-phosphate-based substances in different phases: the intracellular particles and the platelet-like extracellular crystals, likely represent Ca²⁺ accumulated in the vacuoles and crystal-like substances, respectively (FIG. 9).

An elemental analysis using STEM-energy-dispersive X-ray (EDX) spectroscopy shows that both are composed mainly of calcium and phosphorus (FIG. 9). Additionally, an electron diffraction analysis further confirms that the intracellular particles are filled with ACP (FIG. 9) and that the extracellular platelet-like crystals are Hap (FIG. 9). Notably, polyphosphate in vacuoles might also play important roles in maintaining the amorphous form of calcium phosphate. The crystal components are further separated by a gradient centrifugation. The x-ray diffraction (XRD) analysis shows that the HAp produced by the engineered strain was very similar to that of bone. Together with the results based on the fluorescent microscopy, these results indicate that the yeast vacuoles are successfully engineered as devices to transport, accumulate, and store calcium as the form of ACP and the yeast strain to mediate the bone-like HAp synthesis, which is named “osteoyeast”.

### Correlative Imaging Analysis Revealed That Vacuolar ACP Was Secreted as EVs

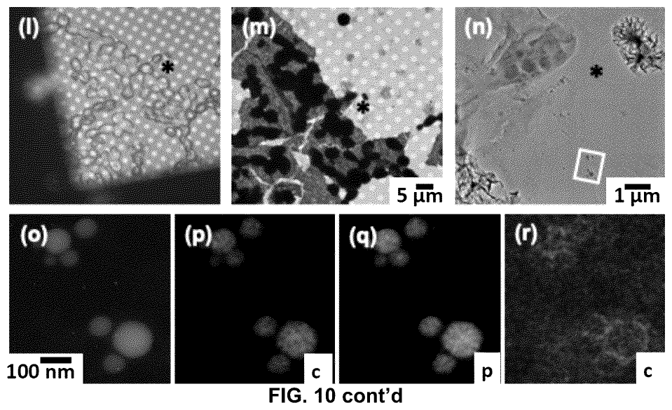

To directly correlate the phenomena observed in the analyses using fluorescence microscopy and TEM, we developed a correlative imaging method to monitor the cellular processes (FIG. 10). This method uses reference/finder TEM grids for locating the same cells. First, we used fluorescence microscopy to monitor the cellular processes for the intracellular ACP formation and the extracellular HAp synthesis. Next, we dried the samples on the grids and analyzed them using TEM. Although the samples moved slightly during drying, relative locations of each cell and HAp crystal corresponded well enough to allow correlation of the two different imaging results.

In the first step, the SB823 strain is grown in the modified YNB media on the TEM grid. Pictures are taken every 45 seconds under the fluorescent microscopy and created into a movie describing the cellular processes. FIG. 10 shows the snapshots for the important events during the HAp synthesis. As expected, the SB823 strain first accumulated the calcein signals within the vacuoles. During this time, small particles are noticed appeared around the yeast cells at 2.083 hr (FIG. 10, panel b arrows). Between 3.760 and 3.823 hr, many HAp-like particles are formed. Notably, fusion of two EVs is observed at 3.785 hr, and this EV is transformed into HAp-like particles at 3.798 hrs. Although the intensity is low, that these particles also found to be fluorescent in the mCherry channel, suggests that they are derived from the vacuoles. Interestingly, the nascent HAp-like particles show the high calcein signals. However, the calcein signals in these particles gradually decreased over time. This may indicate the transformation of ACP into HAp. See FIG. 10.

At 4.748 hr, one of the yeast cells underwent necrosis, and the contents of the cell are exuded. The exudate is also covered with red fluorescence, suggesting that vacuoles were translocated to the outside of cells. This exudate is investigated as a possible mechanism for the HAp synthesis. However, the correlative TEM analysis suggests that the exudate contains ACP rather than HAp. Additionally, the proportion of the yeast cells that undergo necrosis is low, about 1 cell per 200 cells. These results suggest that necrosis is unlikely the primary mechanism for ACP translocation. Further TEM analysis show many EVs about 20 to 150 nm in diameter around the yeast cells. STEM-EDX and electron diffraction reveal that these EVs are filled with ACP. The presence of carbon around each ACP particle suggests that the particles are encapsulated in lipid membranes. The TEM image also shows that several EVs gathered closely. It is possible they may have been about to fuse into larger EVs. See FIG. 10.

Although the molecular mechanisms underlying EV production are yet to be studied, diverse microbes, including fungi and yeasts, are known to produce EVs. Our results suggest that yeast cells have the ability to translocate vacuolar ACP into EVs, which are then secreted. Although it was low, EVs also carry mCherry signals on their membranes. This observation suggests that EVs are associated with vacuoles. However, dilution of red fluorescence may mean that a part of vacuoles might be fused to another organelle or plasma membrane during the process of EV formation. After being secreted, EVs can merge into larger EVs, and ACP in the EVs are transformed into HAp once a certain size is reached or media pH gets high enough to catalyze the transformation of ACP into HAp.

### The Engineered Osteoyeast Platform Enables HAp Synthesis From a Dilute Source of Calcium and Phosphate

The value of our osteoyeast platform is its ability to concentrate calcium and phosphate from dilute sources and transform them into high-value HAp. This ability does not only allow HAp synthesis from the more concentrated sources such as phosphorite, but it would also potentially enable diverse feedstock such as municipal, agricultural, or industrial wastewaters. To prove this, a mock wastewater media containing 1 g/L urea, 1 mM calcium, and 1 mM phosphate is prepared. If the reaction reaches completion, the cells can consume up to 1 mM of calcium and synthesize up to 0.2 mM (100 mg/L) of HAp. The SB818 and SB821 strains are inoculated in this media to a final OD of 0.1. HAp synthesis occurred for xx hr. The calcium concentration is measured every day and found that it did not change in the SB818 culture, but is greatly decreased in the SB820 culture. Analyses identify HAp synthesis only in the SB821 culture. Each culture is also sacrificed and centrifuged to collect the cells and precipitants. After drying the samples at 105° C., their dry weights are determined.

### The Osteoyeast Platform May Enable Production of Materials Based on Other Inorganic Elements

Several interesting phenomena are observed during the TEM analyses of diverse culture samples. The first phenomenon is that a few images suggests that the vacuole can potentially be re-engineered to accumulate metal ions other than calcium. When SB818 is grown in YNB media with a low concentration of calcium (5 mM), TEM analysis shows that the vacuoles were filled with magnesium and potassium. Additionally, when a TEM grid is used with a copper-coated surface, it is observed that SB823 accumulated copper within vacuoles. These observations zre not surprising. because the vacuoles are also thought to be involved in detoxification of toxic metal ions. In addition to the above mentioned metal ions, it was reported that divalent cations Mn²⁺, Fe²⁺, Zn²⁺, Co²⁺, Sr²⁺, and Ni²⁺ and the monovalent cations Li⁺ and Cs⁺ are preferentially sequestered by the vacuoles. While vacuolar transporters may need to be engineered to transport the target metal ions more efficiently, these results suggest that it might be possible to re-engineer the osteoyeast platform to produce diverse nanomaterials.

The second phenomenon encountered was that one analysis suggests the osteoyeast platform could potentially synthesize diverse HAp derivatives. During the correlative analysis, waterproof silicone glue (General Electric Company) is used to adhere the TEM grid to the glass bottom dish as previously described. STEM-EDX analysis reveals that HAp synthesized in the presence of this glue contain silicon in addition to calcium and phosphorus. Synthesis of silicon-substituted HAp (SiHAp) was previously reported, and SiHAp has higher bioactivity compared with regular HAp (33). Because no silicon is observed in the yeast vacuoles, silicon is likely incorporated during the transformation of ACP into HAp. This SiHAp synthesis suggests that the osteoyeast platform allow incorporation of other anions such as carbonate, selenium oxides, bromide, and fluoride to make diverse HAp derivatives and their composites.

### Yeast Cell Surfaces Can Serve as Templates for HAp Synthesis

With osteoblasts, the intracellular ACP particles are secreted from the cells, and they interact with collagen. ACP is gradually transformed into HAp, using collagen as a template. A similar biotemplating mechanism is hypothesized to be involved in S. boulardii-mediated HAp synthesis. At ambient temperatures, pH drift from 4.5 to 6.5 yields mainly dicalcium phosphate (DCP). In the first experiment, an engineered strain is grown in YNB media without CaCl₂. The supernatant of this culture is collected and added 50 mM CaCl₂. This solution yields precipitants. In the subsequent analysis using TEM, the precipitants are identified as ACP. In the second experiment, the wild type strain is grown in YNB media without urea. Upon addition of NaOH to adjust the pH to 7, the culture immediately yields precipitants. The TEM analysis reveals that the precipitants are a composite of yeast cells and HAp. In this composite, multiple yeast cells are densely packed, with thin layers of HAp surrounding them. No ACP formation is observed in these cells. At high pH, Ca²⁺ is known to bind to cell surfaces and mediate flocculation. HAp formation on cell surfaces may share a similar mechanism for mediating flocculation. The results indicate that yeast cell surfaces can serve as templates for HAp formation. The morphology of HAp formed in the above experiment is clearly different from that of HAp synthesized by the microbial process using the engineered strain (FIG. 9). Additionally, significant differences in mechanical properties are identified between the dried precipitants; those from fermentation are powdery, while those from chemical co-precipitation formed ceramic-like frakes that are harder to break into powders. To study how the vacuolar process for ACP accumulation is involved in HAp synthesis, a urease gene cluster in E. coli is overexpressed and performed an ureolytic reaction. As a prokaryote, E. coli does not have vacuoles, the organelle important for transportation, accumulation, and storage of ACP. Therefore, E. coli is useful for investigating what microbial processes take place without vacuole involvement. The precipitants from this reaction are analyzed using TEM. TEM images indicate that the engineered E. coli can make HAp on the outside of their cells, but most of the HAp was crystallized as DCP. These results suggest that vacuoles play crucial roles in HAp synthesis.

### Discussion

Herein is described the successful engineering of a synthetic osteoyeast platform for HAp synthesis, which is guided by the known mechanisms for bone synthesis in osteoblasts. The engineering efforts are initially focused on the vacuoles, a lysosome-like organelle in yeast to develop it as devices for transport, accumulation, and storage of calcium phosphate. The yeast vacuoles are known to play many important roles in maintaining the cellular homeostasis such as degrading proteins, storing metabolites, as well as controlling cytosolic pH and ion concentrations. Additionally, the vacuoles can compartmentalize diverse toxic metals and are involved in their detoxification. In case of calcium, the H⁺-antiporter (VCX1) mediates this process. Therefore, it is believed that increasing cytosolic pH could activate the function of VCX1, which would be enabled by urea decomposition catalyzed by the heterologously expressed ureolytic enzyme and transporter. The engineered strain based on this design principle successfully demonstrates accumulation of calcium phosphate as a form of ACP. Polyphosphate in the vacuoles likely helps maintain ACP as it prevents crystal formation.

As ureolysis proceeded, the yeast cells translocate the vacuolar ACP to EVs and secrete the EVs into the media without further engineering. Many EVs are observed formed around the yeast cells, and their sizes are about 20 to 150 nm in diameters. Although the mechanisms underlying EV formation and secretion are yet to be understood, the ability of yeast cells to secrete ACP in this way might be useful for synthesis of nanocomposites with complex structures. Because of the wide variation in EV diameter, it is likely that these EVs merge and increase in size and volume. ACP inside the EVs is eventually converted into HAp (FIG. 10). Some EVs may also directly interact with pre-existing HAp, and ACP inside the EVs gets converted to HAp using the pre-existing HAp as a template. Because the HAp conversion occurred at similar time points, it may also be triggered by a pH shift. This overall process may be an extension of calcium detoxification and/or pH homeostasis of yeasts. The similarity of this process to the one osteoblasts use for bone synthesis suggests that it might have evolved from this process in ancient eukaryotes.

The ACP in EVs is also maintained by polyphosphate translocated from vacuoles. Polyphosphate is likely depolymerized to monophosphate during the conversion of ACP into HAp. The vacuolar polyphosphatase Ppn1/2 may be responsible for this process. In vacuoles, polyphosphate is produced by the VTC in a process that depends on ATP, and synthesis and degradation of polyphosphate are balanced. However, polyphosphatase activity likely becomes dominant in EVs, because ATP is no longer available for polyphosphate synthesis. While polyphosphate is decomposed by polyphosphatase, some portions may also be replaced with monophosphate in the culture media. This hypothesis is supported by the observation of SiHAp synthesis. The analysis using TEM suggested that some phosphate in HAp synthesized through this osteoyeast platform is replaced by silicon. Because silicon is not found in the vacuoles, silicon is likely incorporated into Hap during the transformation of ACP into HAp. It has been demonstrated that SiHAp has enhanced bioactivity (33). The synthesis of SiHAp also suggests that this platform might be able to synthesize other HAps doped with other anions such as carbonate, selenium oxides, fluoride, and bromide (34).

Finally, it is demonstrated that this platform could produce HAp with high efficiency and yield from a dilute source of calcium and phosphate (both 1 mM). This result suggests that the synthetic osteoyeast platform could tap into cost-effective, abundant, and diverse feedstocks including industrial, agricultural, and residential wastes and wastewater for HAp synthesis. Additionally, this analysis as well as other studies indicate that vacuoles can accumulate diverse metal ions. This phenomenon is very interesting, because it suggests that the platform may be further engineered to produce diverse nanomaterials, including metal phosphate nanoparticles, or to collect heavy metals for bioremediation, and rare metals for biomining. Although these applications would require further optimization of the platform, this synthetic osteoyeast platform offers exciting new opportunities to help create a sustainable bioeconomy.

### Methods

### Strains and Reagents

The Saccharomyces boulardii strain with auxotrophic mutants (trip1, his3, ura3) was provided by the Jin lab (Liu, J. J. et al Metabolic Engineering of Probiotic Saccharomyces boulardii. Applied and Environmental Microbiology, 82 (2016), 2280-2287).

### Cultivation Conditions for HAp Production

Yeast transformations are carried out using the LiAc method. Engineered yeast cells are grown in Yeast Nitrogen Base (YNB) media with appropriate amino acid supplements, optionally supplemented either at the beginning or at a later time point with 20 g/L urea and/or 50 mM CaCl₂. Cells are grown for up to 96 hours at 37° C. Crystals/cells are harvested through a 2.7 μm filter paper, washed with water and left to dry at room temperature. In some instances, cells or cell/crystal suspensions are immediately analyzed without filtering.

### Dry State Transmission Electron Microscopy

Dry pellets containing yeast cells and inorganic materials are dispersed in distilled water and deposited onto lacey carbon 300 mesh copper grids. TEM imaging and selected-area electron diffraction (SAED) were performed at the National Center for Electron Microscopy on a FEI ThemIS TEM equipped with a X-FEG gun and a Ceta2 complementary metal oxide semiconductor (CMOS) camera operating at 300 kV. High-angle annular dark field (HAADF) images are acquired in scanning transmission electron microscopy (STEM) mode at 300 kV with a convergence semi-angle of 11.3 mrad. To determine the compositions of the inorganic materials, energy dispersive X-ray spectroscopy (EDS) is performed using a Bruker SuperX windowless EDS detector, which has a solid angle of 0.7 steradian enabling high count rates with minimal dead time for fast STEM-EDS mapping. STEM-EDS elemental mapping is performed at 300 kV with a 5 min acquisition time.

### Live Cell Fluorescent Microscopy

For long time-lapse fluorescence and bright field microscopy, yeast cells are imaged on sterile glass bottom dishes. Glass-bottom dishes (35 mm with 14 mm #1.5 glass, Cellvis) are treated with 2 mg/mL of Concanavalin A from JackBean (Sigma Aldrich) in 1X PBS and allowed to incubate for 30 minutes. The glass-bottom 1x PBS is then rinsed to remove unbound protein. Yeast cells grown overnight are then diluted in YNB media with or without 50 mM CaCl₂ as described above and allowed to bind to the surface.

Bright field, and fluorescence imaging is performed on an inverted Zeiss Elyra 7 microscope using a Plan-Apo 63x/1.46 NA Oil immersion objective or a Plan-Neofluar 40x/1.3 DIC WD=0.21 M27 objective (Zeiss) and a Sapphire 488 nm (0.5 W), 561 nm (0.5 W) and a Lasos 642 nm (550 mW) laser, a MBS 405/488/561/641 and EF LBF 405/488/561/641 filter set, a LP 560 and a BP 570-620+LP 655 filter cubes. Data are split on a Duolink filter and imaged on 1 or 2 pco.edge 4.2 high speed sCMOS cameras. Images are then analyzed using Zeiss Zen Black, ImageJ or FIJI software.

### Correlative Optical and Electron Microscopy

A 3 mm Au TEM finder grid (Ted Pella) is plasma cleaned in Ar for 20 s then affixed to a glass-bottom dish using ultra-thin (0.0015 in thickness) polyamide tape. The entire dish with the TEM grid is coated with a 2 mg/mL solution of Concanavalin A for 30 min at 37 C. The dish is then washed with 1x PBS before adding 1 uL of the yeast cell culture is transferred onto the petri dish. The dish with the cell culture is incubated for another 30 min at 37 C before the cell media is aspirated off and replaced with crystal growth media. Regions of interest near the alphabet letter markers on the TEM finder grid are selected for live cell imaging. The images are collected in the similar fashion as outlined in the previous section using a Plan-Neofluar 40x to image a wider field of view. At the end of the fluorescent imaging experiment, the media is aspirated and the dish and 1 uL of PBS is gently deposited away from the TEM grids to avoid any excessive turbulent flow while rinsing away the media. After rinsing three times, the polyamide tape is gently removed using sharp tweezers and the TEM grids are allowed to dry in air. After the TEM grids are fully dry, they are loaded into a Thermo Fisher ThemIS TEM and the same regions of interest identified during the optical microscopy experiments are imaged using the protocols described.

### X-Ray Diffraction

Strains are grown in supplemented SC media. After a period of time, the cultures are spun down. Pellets are dried and treated prior to analysis. The dried material is analyzed on a Rigaku MiniFlex 6 XRD.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A genetically modified host cell comprising a first polypeptide capable of active transport of urea into the host cell and/or a second polypeptide capable of degrading urea into ammonia and carbon dioxide, wherein the genetically modified host cell is capable of degrading urea into ammonia and carbon dioxide.

2. The genetically modified host cell of claim 1, wherein first polypeptide capable of active transport of urea into the host cell is a urea active transporter.

3. The genetically modified host cell of claim 2, wherein the urea active transporter is a Saccharomyces boulardii or S. cerevisiae urea active transporter.

4. The genetically modified host cell of claim 1, wherein second polypeptide capable of degrading urea into ammonia and carbon dioxide is a urea amidolyase or a urease.

5. The genetically modified host cell of claim 4, wherein the urea amidolyase is a S. boulardii or S. cerevisiae urea amidolyase.

6. The genetically modified host cell of claim 1, wherein genetically modified host cell comprises a first nucleic acid encoding the first polypeptide operatively linked to a first promoter capable of expressing the first polypeptide in the genetically modified host cell.

7. The genetically modified host cell of claim 1, wherein the genetically modified host cell comprises a second nucleic acid encoding the second polypeptide operatively linked to a second promoter capable of expressing the second polypeptide in the genetically modified host cell.

8. The genetically modified host cell of claim 1, wherein genetically modified host cell is modified to reduce or eliminate nitrogen catabolite repression with the host cell.

9. The genetically modified host cell of claim 1, wherein the genetically modified host cell is a Saccharomyces cell is S. boulardii or S. cerevisiae.

10. A culture medium comprising the genetically modified host cell of claim 1, wherein culture medium comprises a urea, a calcium salt or calcium ion, and a phosphate.

11. A method for producing calcium phosphate, and/or a calcium phosphate biocomposite material, the method comprising: (a) providing a genetically modified host cell of claim 1 in a medium comprising urea, a calcium salt or calcium ion, and a phosphate, and (c) culturing the genetically modified host cell to produce ammonia such that calcium phosphate is produced.

12. The method of claim 11, wherein the calcium phosphate produced is amorphous calcium phosphate (ACP) particles and/or hydroxyapatite (HAP) crystals.

13. The method of claim 12, wherein the ACP particles are intracellular and the HAP crystals are extracellular and/or platelet-like.

14. The method of claim 12, wherein the method further comprises shifting the pH of the medium.

15. The method of claim 11, wherein the shifting comprises adding to the medium or mixing with the medium a base to shift the pH up, such that intracellular amorphous calcium phosphate (ACP) particles and/or hydroxyapatite (HAP) crystals are produced.
